SEQUENCE LISTING (18 sequences)

SEQ ID NO: 1 - DNA, 2262 bp, Glycine max
tcaacaataggcttacaggttccatacctctttctttaggtttctgccctttgcttcagtctcttgacctcagcaacaacttgctcacaggagcaatcccttatagtcttgctaattccactaagctttattggcttaacttgagtttcaactccttctctggtcctttaccagctagcctaactcactcattttctctcacttttctttctcttcaaaataacaatctttctggctcccttcctaactcttggggtggaaattccaagaatggcttctttaggcttcaaaatttgatcctagataataactttttcactggtgacgttcctgcttctttgggtagcttaagagagctcaatgagatttcccttagtcataataagtttagtggagctataccaaatgaaataggaaccctttctaggcttaagacacttgacatttctaataatgccttgaatgggaacttgcctgctaccctctctaatttatcctcacttacactgctgaatgcagagaacaacctccttgacaatcaaatccctcaaagtttaggtagattgcgtaatctttctgttctgattttgagtagaaaccaatttagtggacatattccttcaagcattgcaaacatttcctcgcttaggcagcttgatttgtcactgaataatttcagtggagaaattccagtctccttcgacagtcagcgcagtctaaatctcttcaatgtttcctacaatagcctttcaggttctgttccccctctgcttgccaagaaatttaactcaagctcatttgtgggaaatattcaactatgtgggtacagcccttcaaccccatgtctttcccaagctccatcacaaggagtcattgccccacctcctgaagtgtcaaaacatcaccatcataggaagctaagcaccaaagacataattctcatagtagcaggagttctcctcgtagttctgattatactttgttgtgtcctgcttttctgcctgatcagaaagagatcaacatctaaggccgggaacggccaagccaccgagggtagagcggccactatgaggacagaaaaaggagtccctccagttgctgctggtgatgttgaagcaggtggggaggctggagggaaactagtccattttgatggaccaatggcttttacagctgatgatctcttgtgtgcaacagctgagatcatgggaaagagcacctatggaactgtttataaggctattttggaggatggaagtcaagttgcagtaaagagattgagggaaaagatcactaaaggtcatagagaatttgaatcagaagtcagtgttctaggaaaaattagacaccccaatgttttggctctgagggcctattacttgggacccaaaggggaaaagcttctggtttttgattacatgtctaaaggaagtcttgcttctttcctacatggtaagtttcgtgtgctgttctttcattaagtgttgtgtgtgctgttctttaattataatttggagttttaccttagtaatctgtataattctaatcggagaacagtacaaacaaaaacacctaaggaacactatagcacctaaggaacaacaccttagctttaatataccatatcaataagtgaattattttcttgttcatcttgatgcaggtggtggaactgaaacattcattgattggccaacaaggatgaaaatagcacaagacttggcccgtggcttgttctgccttcattcccaggagaacatcatacatgggaacctcacatccagcaatgtgttgcttgatgagaatacaaatgctaaaattgcagattttggtctttctcggttgatgtcaactgctgctaattccaacgtgatagctacagctggagcattgggataccgggcacctgagctctcaaagctcaagaaagcaaacactaaaactgatatctacagtcttggtgttatcttgttagaactcctaacgaggaaatcacctggggtgcctatgaatggactagatttgcctcagtgggttgcctcagttgtcaaagaggagtggacaaatgaggtttttgatgcagacttgatgagagatgcatccacagttggcgacgagttgctaaacacgttgaagctcgctttgcactgtgttgatccttctccatcagcacgaccagaagttcatcaagttctccagcagctggaagagattagaccagagagatcagtcacagccagtcccggggacgatatcgtatag

SEQ ID NO: 2 - DNA, 2240 bp, Glycine max
gaaatgctttgcttgggaaaccccttagtcctggaggtggaccaagtggaactactccttctgggtcttcgaccggtggaagtggtggtgaatcctcaaagggtaattcttcggtgtcgccaggttggattgctggtatagttgttattgtgttgttttttattgcagtggtgttgtttgtgtcttggaagtgttttgtcaacaagctgcaggggaagttcagtagggttaaaggtcatgaaaatgggaaaggaggctttaaacttgatgctgtccatgtttctaatggatatggtggtgttccagttgagttgcaaagccagagcagtggtgatcgcagtgaccttcatgctttagatggtccaacattttctatccaagttcttcgacaagtgacgaataatttcagcgaggagaacattttaggcaggggagggtttggagtagtttataagggggtgttgcatgatggaacaaaaattgctgttaagaggatggaatctgttgcaatggggaacaaaggtcagaaagagttcgaagcagagattgcacttcttagtaaagttaggcatagacatttggttgctcttctagggtattgcatcaatggcaatgaaaggcttttggtgtatgagtatatgcctcaaggtacattaacacagcacctgtttgagtggcaggagcatgggtatgctcctttgacttggaagcaaagggtagtaatagctttggatgtagcgcggggggtggaatacttgcacagtttagctcagcaaagcttcattcctagagacttaaaaccctcaaacatactactaggcgatgacatgagagcaaaggttgctgattttgggttggttaaaaatgcaccagatgggaagtattctgttgagacacggttggctggaacatttggatatcttgcacctgagtatgcaggtacagaaagcctttgattttagttttgtacaattgtgccttaattttgaagttcatattttatatgctcgtatttggtggttatagctgttggttattacttcaatatcatgcttcggtgttcagcaaatttaagtagttcaccagagtaatcgctcacatacaaaaaaaaagtagaaagagttgaagggaaaataattgatactcaattcctagatacatggctacttcaaaattctttgtggctatttctttgcaatgttatattttgctcttttcacgtgttttgttgagttgggtgggggttttgctgcatagttcttggtggttgatgcctcaaaagatatgtcgagccatttttagacagtttaccagagtctgactctcaattatcctttacgatgtgatgaatactctggttgcattaaatctttgattgctgtatattgtcatgggcttaggtggtagttctctgtcgcaactaatcactgttgtggaatttatcattctatcccatttccttgttggatcggtgcattgaaacatcttttgttaaaactgttattttgatcggtgttgtttttatccatttagcatcaagacttttgcaagcataaaacttcctaaaatgttgcattgaatgtgattagatggcatttgattagtgctagtctatttgtttgattatatttaatgttactctgtttcttaccagctactggaagagtgacaaccaaagtggatgtttatgcatttggagtagttctgatggaacttatcaccggtagaaaggcattggatgatactgtgccagatgaaaggtctcacttggtgacatggttccgtagggtactaattaacaaggaaaacattccaaaggcaattgatcaaattctcaatccagatgaggaaaccatgggaagcatatatacagtggccgagctggcaggccattgcactgctcgcgaaccataccaaaggccggatatgggtcatgcagtgaacgtcttggttcctcttgtggagcaatggaaacctactagccatgatgaagaagaggaagacggctctggcggtgaccttcatatgagccttcctcaagctctacgaaggtggcaagccaacgaaggcacttcctcaatatttaatgacatttccatctcacaaacccaatcaagcatctcctctaaacctgcagggtttgcagactcctttgattcaatggattgccgttaaccaagggcgaattcgtttaaacctgcaggactagtccctttagtgagggttaattctgagcttgg

SEQ ID NO: 3 - DNA, 25 bp, Artificial sequence
  Other information Description of artificial sequence synthetic primer
ccagaccctgtttaaatttcctttt

SEQ ID NO: 4 - DNA, 23 bp, Artificial sequence
  Other information Description of artificial sequence synthetic primer
acgtggtgaggaaggagctaact

SEQ ID NO: 5 - DNA, 23 bp, Artificial sequence
  Other information Description of artificial sequence synthetic primer
ttctggctcccttcctaactctt

SEQ ID NO: 6 - DNA, 25 bp, Artificial sequence
  Other information Description of artificial sequence synthetic primer
aattttgaagcctaaagaagccatt

SEQ ID NO: 7 - DNA, 21 bp, Artificial sequence
  Other information Description of artificial sequence synthetic primer
cggccactatgaggacagaaa

SEQ ID NO: 8 - DNA, 18 bp, Artificial sequence
  Other information Description of artificial sequence synthetic primer
cagcctccccacctgctt

SEQ ID NO: 9 - DNA, 29 bp, Artificial sequence
  Other information Description of artificial sequence synthetic primer
catattttatatgctcgtatttggtggtt

SEQ ID NO: 10 - DNA, 20 bp, Artificial sequence
  Other information Description of artificial sequence synthetic primer
tgctgaacaccgaagcatga

SEQ ID NO: 11 - DNA, 16 bp, Artificial sequence
  Other information Description of artificial sequence synthetic probe
tcttgcaagtgtctca

SEQ ID NO: 12 - DNA, 17 bp, Artificial sequence
  Other information Description of artificial sequence synthetic probe
ctcttgctagtgtctca

SEQ ID NO: 13 - DNA, 16 bp, Artificial sequence
  Other information Description of artificial sequence synthetic probe
cttggaatttccaccc

SEQ ID NO: 14 - DNA, 15 bp, Artificial sequence
  Other information Description of artificial sequence synthetic probe
tggaattcccacccc

SEQ ID NO: 15 - DNA, 15 bp, Artificial sequence
  Other information Description of artificial sequence synthetic probe
tgctgctggtgatgt

SEQ ID NO: 16 - DNA, 15 bp, Artificial sequence
  Other information Description of artificial sequence synthetic probe
tgctggtggtgatgt

SEQ ID NO: 17 - DNA, 18 bp, Artificial sequence
  Other information Description of artificial sequence synthetic probe
tgttggttagtacttcaa

SEQ ID NO: 18 - DNA, 18 bp, Artificial sequence
  Other information Description of artificial sequence synthetic probe
tagctgttggttattact

(19) United States
(12) Patent Application Publication
(10) Pub. No.: US 2009/0100537 A1
(43) Pub. Date: Apr. 16, 2009

(54) METHODS AND COMPOSITIONS FOR HIGH YIELDING SOYBEANS WITH NEMATODE RESISTANCE

(76) Inventors: Vergel Concibido, Maryland Heights, MO (US); Holly Kleiss, Auburn, IL (US); Jennifer Hicks, Ames, IA (US); James Narvel, Middletown, DE (US); Nancy Sebern, Marshalltown, IA (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 12/246,535

(22) Filed: Oct. 7, 2008

Related U.S. Application Data
(60) Provisional application No. 60/979,422, filed on Oct. 12, 2007.

(51) Int. Cl. C12Q 1/68; A01H 1/00; A01H 5/00

(52) U.S. Cl. 800/265; 800/312; 800/300; 800/302; 800/301; 435/6

(57) ABSTRACT

The present invention is in the field of plant breeding and host plant resistance. More specifically, the invention provides methods to evaluate and select soybean plants that exhibit resistance to multiple races of nematodes in addition to yield parity and an agronomically phenotype. The invention provides methods and compositions for selecting and introgressing resistant alleles to obtain nematode resistant soybeans with yield parity.

### CROSS-REFERENCE TO RELATED APPLICATIONS

This application claims the benefit under 35 U.S.C. §119(e) of U.S. Provisional Application No. 60/979,422 filed Oct. 12, 2007. The entirety of the application is hereby incorporated by reference.

### INCORPORATION OF THE SEQUENCE LISTING

A sequence listing containing the file named “pa_{—}55015B.txt”, which is 10,664 bytes) (as measured in Microsoft Windows®) and created on Sep. 23, 2008, comprises 18 nucleotide sequences. This electronic sequence listing is electronically filed herewith and is incorporated herein by reference.

### BACKGROUND OF THE INVENTION

1. Field of the Invention

The present invention is in the field of plant breeding. More specifically, the invention provides methods and compositions to select for and generate soybean plants that exhibit resistance to multiple races of nematodes in addition to a yield parity and agronomically elite phenotype.

2. Description of Related Art

Soybean cyst nematode (SCN) (Heterodera glycines Ichinohe), is the most destructive pest of soybean [Glycine max (L.) Merrill]. In the US alone, yield losses in 2002 attributed to SCN were estimated at 3.6 million megagrams, resulting in about $783.8 million (Wrather et al. Plant Health Progress doi:10.1094/PHP-2003-0325-01-RV, 2003). However, host plant resistance is a cost-effective and low input method of controlling SCN. Soybean cyst nematode-resistant cultivars yield better at SCN-infested sites but lose this superiority to susceptible soybean cultivars at non-infested sites (Donald et al. Journal of Nematology. 38: 76-82, 2006). The widespread adoption of SCN resistant varieties has been hampered due to lower yields of SCN-resistant varieties compared to susceptible varieties under low SCN pressure.

One hundred and eighteen plant introductions (PIs) and wild species are currently known to be resistant to SCN. Of the SCN resistant cultivars developed in the USA, resistance can be traced to five sources: G. max ‘Peking’, PI 88788, PI 90763, PI 437654, or PI 209332. The predominant source of SCN resistance in the midwestern USA is PI 88788, though a few cultivars have been released with resistance from PI 90763, PI 437654, and PI 209332. Across North America, more than 90% of all SCN resistant varieties carry PI 88788-derived resistance. This is brought about by the widespread use of the cultivar “Fayette” as a source of PI 88788, whose popularity is due to good agronomic characteristics.

Molecular marker technology has facilitated the identification and characterization of quantitative trait loci (QTL) underlying SCN resistance. In almost all QTL mapping studies, two loci, rhg1 on linkage group (LG) G and Rhg4 on LG A2, appeared to be the most important and most common among various sources of resistance. PI 437654, PI 209332, PI 88788, PI 90763, PI 89772, and Peking all have the major SCN resistance gene, rhg1, on linkage group G (Cregan et al, TAG 99: 811-818, 1999). This locus controls a large portion of the total variation for resistance and is effective against several different population types of SCN. In addition, Peking, PI 209332, and PI 437654 have the resistance gene Rhg4 that maps near the I locus (black seed-coat pigmentation) on linkage group A2 (Cregan et al. TAG 99: 811-818, 1999). In P188788, resistance appeared to be controlled mostly by rhg1 and additional effects are contributed by Rhg4 and Rhg5 against other SCN populations. Two other genes Rhg2 and Rhg3 have been postulated but have not been confirmed and characterized. However, in Peking, SCN resistance is bigenic and requires both rhg1 and Rhg4 to have complete resistance to race 3 (HG 0, HG 7). Either gene used singly is not effective at providing plant protection against SCN regardless of race or isolate.

The yield deficit associated with introgression of SCN resistance in soybeans is well-documented. SCN resistant soybean cultivars yield 5 to 10% less than susceptible cultivars when grown in environments with low SCN pressure (Noel, Biology and management of the soybean cyst nematode, APS Press, St. Paul, Minn. p. 1-13, 1992). The yield deficit in cultivars with SCN resistance alleles derived from PI 88788 was on average 161 kg [ha.sup. −1] less than susceptible cultivars in noninfested field trials (Chen et al. Plant Dis Vol. 85: 760-777, 1999).

Linkage between SCN resistance and reduced yield was also reported by Mudge et al. (Soybean Genet News1 23:175-178, 1996). In their study, populations segregating for SCN resistance derived from G. max PI 209332 revealed yield reducing quantitative trait loci (QTL) alleles in coupling linkage with the SCN resistance gene rhg1. These yield reducing alleles mapped approximately 10 cM from each other and a difference of 296 kg/ha for the QTL distal to rhg1 and 632 kg/ha for the QTL proximal to rhg1 was measured when homozygous resistant and susceptible lines were compared. This region was also associated with an increase in height and lodging, later maturity, and a decrease in seed protein and oil content.

Kopisch-Obuch et al. (Crop Sci 45:956-965, 2005) tested for linkage between SCN resistance and reduced yield in near isogenic line (NIL) populations developed from soybean cultivars with resistance derived from G. max PI 88788. Five NIL populations were segregating for resistance at rhg1 and two populations were segregating for resistance at cqSCN-003 locus on LG J. In multiple field studies at locations with low SCN pressure, NILs carrying the SCN resistance allele yielded significantly (P<0.05) less (118 kg/ha) than NILs carrying the susceptible alleles in one population segregating for rhg1 and in one population segregating for cqSCN-003 locus (76 kg/ha). Molecular marker analysis of the regions flanking the resistance genes suggested the presence of a yield reducing allele distal to rhg1 and possibly another yield reducing allele linked or pleiotropic to cqSCN-003 locus. In several populations, an association between SCN resistance with maturity, height, and lodging was measured, but differences were small in magnitude.

The yield deficit associated with SCN resistance in noninfested or low SCN pressure environments can be attributed to pleiotropic effects of the SCN resistance gene(s) on yield or linkage and coinheritance of genes effecting yield. There is a need in the art for a system to manage SCN pest pressure without a yield penalty.

The present invention provides a method to evaluated soybeans in low and high SCN infested areas under field conditions. The density of the SCN populations is maintained over time through a series of crop rotation and catch crops. The prior art has failed to provide a field assay to evaluate yield in conjunction with SCN resistance.

The invention overcomes deficiencies of the prior art by providing methods and compositions for selecting and generating soybean plants exhibiting yield parity when cultivated under low to non-infested SCN fields. More specifically, the invention provides methods and compositions for selecting and introgressing alleles of rhg1 and Rhg4 from ‘Forrest,’ derived from Peking, to produce a high yielding SCN resistant soybean, regardless of SCN infestation pressure. The prior art has failed to provide SCN resistant soybean varieties that exhibits yield parity when cultivated under conditions low to non-infested. However, there is a great need for such soybean plants.

### SUMMARY OF THE INVENTION

The present invention includes a method of soybean breeding for yield parity to susceptible plants and SCN resistant plants irrespective of SCN infestation levels comprising: (A) crossing a first soybean having Forrest-type SCN resistance with a second soybean to create a segregating population; (B) selecting at least one soybean plant comprising the Forrest-type SCN resistant alleles of rhg1 and Rhg4. Moreover, the present invention relates to producing SCN resistant plants, populations, lines, lines, and varieties that exhibit at least yield parity. Furthermore, the present invention relates to producing SCN resistant plants capable of producing grain yield comprising equal to, 5% higher than, 10% higher than, 15% higher than susceptible plants.

More particularly, the present invention includes a method of introgressing an Forrest-type rhg1 and Rgh4 alleles into a soybean plant comprising (A) crossing at least one first soybean plant comprising a nucleic acid molecule selected from the group consisting of SEQ ID NO: 1 through SEQ ID NO: 2 with at least one second soybean plant in order to form a segregating population, (B) screening the segregating population with one or more nucleic acid markers to determine if one or more soybean plants from the segregating population contains the nucleic acid molecule, and (C) selecting from the segregating population one or more soybean plants comprising a nucleic acid molecule selected from the group consisting of SEQ ID NO: 1 through SEQ ID NO: 2.

The present invention includes a method of soybean breeding for yield parity to commercial check varieties and SCN resistant plants irrespective of SCN infestation levels comprising: (A) crossing a first soybean having Forrest-type SCN resistance with a second soybean to create a segregating population; (B) selecting at least one soybean plant comprising the Forrest-type SCN resistant alleles of rhg1 and Rhg4. Moreover, the present invention relates to producing SCN resistant plants, populations, lines, lines, and varieties that exhibit at least yield parity. Furthermore, the present invention relates to producing SCN resistant plants capable of producing grain yield comprising equal to, 5% higher than, 10% higher than, 15% higher than commercial check varieties plants.

More particularly, the present invention includes a method of introgressing an Forrest-type rhg1 and Rgh4 alleles into a soybean plant comprising (A) crossing at least one first soybean plant comprising a nucleic acid molecule selected from the group consisting of SEQ ID NO: 1 through SEQ ID NO: 2 with at least one second soybean plant in order to form a segregating population, (B) screening the segregating population with one or more nucleic acid markers to determine if one or more soybean plants from the segregating population contains the nucleic acid molecule, and (C) selecting from the segregating population one or more soybean plants comprising a nucleic acid molecule selected from the group consisting of SEQ ID NO: 1 through SEQ ID NO: 2.

In a preferred embodiment, the present invention further provides soybean plants that further comprises a transgenic trait, wherein the transgenic trait may confers to the soybean plant a preferred property selected from the group consisting of herbicide tolerance, increased yield, insect control, fungal disease resistance, virus resistance, nematode resistance, bacterial disease resistance, mycoplasma disease resistance, altered fatty acid composition, altered oil production, altered amino acid composition, altered protein production, increased protein production, altered carbohydrate production, germination and seedling growth control, enhanced animal and human nutrition, low raffinose, drought and/or environmental stress tolerance, altered morphological characteristics, increased digestibility, industrial enzymes, pharmaceutical proteins, peptides and small molecules, improved processing traits, improved flavor, nitrogen fixation, hybrid seed production, reduced allergenicity, biopolymers, biofuels, and any combination of these.

The present invention includes a method of identifying a haplotype for rhg1 from ‘Forrest’ associated with yield parity and SCN resistance comprising: (A) genotyping at least one single nucleotide polymorphisms (SNP) in the rhg1 region in at least two soybean plants; (B) determining the yield and SCN resistance values for the plants; (C) identifying at least two haplotypes in the rhg1 region associated with yield parity and SCN resistance; (D) selecting at least one soybean plant comprising the haplotype associated with yield parity and SCN resistance. Moreover, the present invention relates to producing SCN resistant plants, populations, lines, lines, and varieties that exhibit at least yield parity. More particularly, the present invention includes a method of introgressing an rhg1 and Rgh4 from ‘Forrest’ into a soybean plant comprising (A) crossing at least one first soybean plant comprising a nucleic acid molecule selected from the group consisting of SEQ ID NO: 1 through SEQ ID NO: 2 with at least one second soybean plant in order to form a segregating population, (B) screening the segregating population with one or more nucleic acid markers to determine if one or more soybean plants from the segregating population contains the nucleic acid molecule, and (C) selecting from the segregating population one or more soybean plants comprising a nucleic acid molecule selected from the group consisting of SEQ ID NO:1 through SEQ ID NO: 2.

Plants containing one or more SCN resistant loci described can be donor plants. Soy plants containing resistant loci can be, for example, screened for by using a nucleic acid molecule capable of detecting a marker polymorphism associated with resistance. In one aspect, a donor plant is MV00045 (Budapest Treaty Deposit Number at PTA-8740). In a preferred aspect, a donor plant is the source for SCN resistance loci 1 and 2. In another preferred aspect, a donor plant is the source for SCN resistance locus 1. A donor plant can be a susceptible line. In one aspect, a donor plant can also be a recipient soy plant.

Furthermore, the present invention provides a method for assaying at least one soybean plant for yield and susceptibility, partial resistance or resistance to SCN comprising the steps of: (A) maintaining a field nursery with low densities of SCN, (B) maintaining a field nursery with high densities of SCN, (C) cultivating the plant in the low and high SCN field nursery, (D) assessing the plant for susceptibility, partial resistance or resistance to SCN, and (E) assessing the plant for yield. In a preferred embodiment, the present invention further provides soybean plants that exhibit yield parity when cultivated under conditions selected from the group consisting non-infested, low, moderate, and high nematode pressure, with resistance to nematodes, including, but not limited to Heterodera sp. such as soybean cyst nematode (Heterodera glycines), Belonolaimus sp. such as sting nematode (Belonolaimus longicaudatus), Rotylenchulus sp. such as reniform nematode (Rotylenchulus reniformis), Meloidogyne sp. such as southern root-knot nematode (Meloidogyne incognita), peanut root-knot nematode (Meloidogyne arenaria) and the Javanese root-knot nematode (Meloidogyne javanica).

Moreover, the present invention relates to a method of promoting a soybean variety capable of nematode resistance and high yield. The method comprises providing information that a nematode resistant soybean is capable of high yield irrespective of nematode infestation pressure. Furthermore, the method provides information comprises the origin of nematode resistance, wherein the origin is “Forrest”, “Peking” or “Accomac”. Additionally, the method disseminates information by oral or visual medium selected from the group consisting of television, film, video, radio, extension presentations, oral presentations, print, newspapers, magazines, technical bulletins, extension bulletins, packaging, seed bags, bag tags, brochures, photography, electronic, internet, blogs and e-mail.

### BRIEF DESCRIPTION OF NUCLEIC ACID SEQUENCES

SEQ ID NO: 1 is a genomic sequence derived from Glycine max (L.) Merrill corresponding to rhg1 .

SEQ ID NO: 2 is a genomic sequence derived from Glycine max (L.) Merrill corresponding to Rhg4.

SEQ ID NO: 3 is a PCR primer for amplifying SEQ ID NO: 1.

SEQ ID NO: 4 is a PCR primer for amplifying SEQ ID NO: 1.

SEQ ID NO: 5 is a PCR primer corresponding to SEQ ID NO: 1.

SEQ ID NO: 6 is a PCR primer corresponding to SEQ ID NO: 1.

SEQ ID NO: 7 is a PCR primer corresponding to SEQ ID NO: 1.

SEQ ID NO: 8 is a PCR primer corresponding to SEQ ID NO: 1.

SEQ ID NO: 9 is a PCR primer corresponding to SEQ ID NO: 2.

SEQ ID NO: 10 is a PCR primer corresponding to SEQ ID NO: 2.

SEQ ID NO: 11 is a first probe for detecting the nematode resistance allele of SEQ ID NO: 1.

SEQ ID NO: 12 is a second probe for detecting the nematode resistance allele of SEQ ID NO: 1.

SEQ ID NO: 13 is a first probe corresponding to the nematode resistance allele of SEQ ID NO: 1.

SEQ ID NO: 14 is a second probe corresponding to the nematode resistance allele of SEQ ID NO: 1.

SEQ ID NO: 15 is a first probe corresponding to the nematode resistance allele of SEQ ID NO: 1.

SEQ ID NO: 16 is a second probe corresponding to the nematode resistance allele of SEQ ID NO: 1.

SEQ ID NO: 17 is a first probe corresponding to the nematode resistance allele of SEQ ID NO: 2.

SEQ ID NO: 18 is a second probe corresponding to the nematode resistance allele of SEQ ID NO: 2.

### DESCRIPTION OF FIGURES

FIG. 1. Maintenance for field nurseries with high densities of SCN. The plot is divided into quadrants and planted with test material, corn and two quadrants of herbicide susceptible and SCN susceptible soybean. The crops are rotated within the site each season.

FIG. 2. Maintenance for field nurseries with low densities of SCN. The plot is divided into quadrants and planted with test materials, “catch” soybean (herbicide and SCN susceptible soybean), and two quadrants of corn. The “catch” is sprayed with an herbicide to kill the soybean host and reduce SCN numbers. In addition, quadrant with “catch” is planted to wheat, oat to reduce fallow syndrome. The crops are rotated within the site each season.

FIG. 3: SCN resistant soybeans with both rhg1 and Rhg4 derived from ‘Forrest’ have a yield benefit compared with other SCN resistant soybeans. rhg1 -8 indicates rhg1 derived from PI 88788. rhg1-P indicates rhg1 from ‘Forrest. Rhg4-P indicates Rhg4 from ‘Forrest’. S indicates Rhg4 is absent.

FIG. 4: SCN resistant soybeans with both rhg1 and Rhg4 derived from ‘Forrest’ have a yield benefit compared with other SCN susceptible soybeans. A population was developed by crossing MV0046 with MV0045. MV0045 was the source of resistance derived from ‘Forrest’. The progeny were genotyped for the rhg1 haplotypes and presence of Rhg4.

### DETAILED DESCRIPTION OF THE INVENTION

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 3^{rd} Edition, Garland Publishing, Inc.: New York, 1994; Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR §1.822 is used.

As used herein, “marker” means polymorphic sequence. A “polymorphism” is a variation among individuals in sequence, particularly in DNA sequence. Useful polymorphisms include single nucleotide polymorphisms (SNPs), insertions or deletions in DNA sequence (Indels) and simple sequence repeats of DNA sequence (SSRs).

As used herein, “marker assay” means a method for detecting a polymorphism at a particular locus using a particular method, e.g. phenotype (such as seed color, flower color, or other visually detectable trait), restriction fragment length polymorphism (RFLP), single base extension, electrophoresis, sequence alignment, allelic specific oligonucleotide hybridization (ASO), RAPD, etc.

As used herein, the term “single nucleotide polymorphism,” also referred to by the abbreviation “SNP,” means a polymorphism at a single site wherein said polymorphism constitutes a single base pair change, an insertion of one or more base pairs, or a deletion of one or more base pairs.

As used herein, the term “haplotype” means a chromosomal region within a haplotype window defined by at least one polymorphic molecular marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in our definition of what constitutes a haplotype so long as the functional integrity of that genomic region is unchanged or improved.

As used herein, the term “haplotype window” means a chromosomal region that is established by statistical analyses known to those of skill in the art and is in linkage disequilibrium. Thus, identity by state between two inbred individuals (or two gametes) at one or more molecular marker loci located within this region is taken as evidence of identity-by-descent of the entire region. Each haplotype window includes at least one polymorphic molecular marker. Haplotype windows can be mapped along each chromosome in the genome. Haplotype windows are not fixed per se and, given the ever-increasing density of molecular markers, this invention anticipates the number and size of haplotype windows to evolve, with the number of windows increasing and their respective sizes decreasing, thus resulting in an ever-increasing degree confidence in ascertaining identity by descent based on the identity by state at the marker loci.

As used herein, “genotype” means the genetic component of the phenotype and it can be indirectly characterized using markers or directly characterized by nucleic acid sequencing. The genotype may constitute an allele for at least one marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome.

As used herein, “phenotype” means the detectable characteristics of a cell or organism which are a manifestation of gene expression.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, “linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. Two loci are said to be “genetically linked” when they are in linkage disequilibrium.

As used herein, “quantitative trait locus (QTL)” means a locus that controls to some degree numerically representable traits that are usually continuously distributed.

As used herein, “resistance allele” means the isolated nucleic acid sequence that includes the polymorphic allele associated with resistance to soybean cyst nematode.

As used herein, the term “soybean” means species Glycine max, Glycine soja or any species that is sexually compatible with Glycine max.

As used herein, the term “elite line” means any line that has resulted from breeding and selection for superior agronomic performance. An elite plant is any plant from an elite line.

As used herein, the term “soybean cyst nematode” or “SCN” refers to Heterodera glycines.

As used herein, the term “biotype” or “isolate” refers to the classification of an SCN population based on the race test or the HG-test.

As used herein, the term “non pressure” or “non infestation” refers to 0 SCN eggs/100 cc soil.

As used herein, the term “low pressure” or “low infestation” refers to 1 to 500 SCN eggs/100 cc soil.

As used herein, the term “moderate pressure” or “moderate infestation” refers to 500 to 2000 eggs/100 cc soil.

As used herein, the term “high pressure” or “high infestation” refers to greater than 2000 eggs/100 cc soil.

As used herein, the term “high yielding nematode resistant plant” or “high yielding” refers to a soybean plant that produces a commercially significant yield in one or more specific plantings when cultivated under low nematode pressure.

As used herein, the term “commercially significant yield” or “agronomically acceptable yield” refers to a grain yield of at least 100% of a check variety such as AG2703 or DKB23-51.

As used herein, the term “yield parity” means equivalency in yield to that of a check variety such as AG2703 or DKB23-51 when cultivated in more than one environment.

As used herein, the term “high yield” refers to a grain yield at least 103% of a check variety such as AG2703 or DKB23-51.

As used herein, the term “fallow syndrome” refers to a condition that can severely limit the plant growth. Young root systems are colonized by vesicular arbuscular mycorrhizae, which assist in nutrient uptake. The mycorrhizae population is substantially reduced when non-host crops, such as sugarbeet or canola, or fallow precedes soy in rotation. Planting of host crops, such as oat or wheat, can increase the mycorrhizae population and reduce the effects of fallow syndrome.

As used herein, the term “Forrest-type” resistance refers resistance derived from the cv. Forrest which carries resistance from Peking.

As used herein, the term “comprising” means “including but not limited to”.

The present invention overcomes deficiencies of the prior art by providing agronomically soybean varieties that exhibit nematode resistance and yield parity when cultivated under no, low, moderate or high nematode pressure. The invention is significant because SCN resistant soybean varieties generally have a yield deficit compared to susceptible commercial check varieties when cultivated under non-infested and low pressure. SCN resistant soybean varieties only have a yield benefit, as compared to susceptible commercial cultivars, when cultivated under moderate to high pressure. It has been estimated that SCN resistant soybean yield 5-10% less than susceptible soybeans cultivated in low SCN pressure environments (Noel, Biology and management of the soybean cyst nematode, APS Press, St. Paul, Minn. p. 1-13, 1992). The present invention provides SCN resistant soybean plants that exhibit at least yield parity when cultivated under no, low, moderate or high SCN pressure.

The provision of nematode resistance in conjunction with desirable agronomic characteristics, such as yield parity under low pressure, provides many benefits and provides a desirable product concept for farmers wanting to mitigate disease risk without compromising on yield. SCN is a destructive pest of soybean. Host plant resistance is a cost-effective and low input method of controlling SCN, however, the widespread adoption of SCN resistant varieties has been hampered due poor yields under low SCN pressure.

The present invention provides genetic markers and methods for use in the generation of improved plants. Rhg4 and rhg1 have been sequenced (U.S. Pat. No. 7,154,021). Diagnostic SNP markers were developed from the sequence information to identify and assist in the introgression of rhg1 derived from different resistant source, including Peking and PI 88788, and Rhg4 derived from Peking.

The rhg1 locus is located on linkage group G. In the present invention, SNP markers used to monitor the introgression of rhg1 include SED ID NO: 1. Illustrative SNP marker DNA molecule (SEQ ID NO: 1) can be amplified using the primers indicated as SEQ ID NO: 3 through SEQ ID NO: 8 with probes indicated as SEQ ID NO: 11 through SEQ ID: 16. In the present invention, Rhg4 is located on linkage group A2. A SNP marker used to monitor the introgression of Rhg4 derived from Peking is SEQ ID NO: 2. Illustrative SNP marker DNA molecule (SEQ ID NO: 2) can be amplified using the primers indicated as SEQ ID NO: 9 through SEQ ID: 10 with probes indicated as SEQ ID NO: 17 through SEQ ID 18.

The present invention also provides a soybean plant comprising a nucleic acid molecule selected from the group consisting of SEQ ID NO: 1 and SEQ ID: NO: 2 and complements thereof. The present invention also provides a soybean plant comprising a nucleic acid molecule the group consisting of SEQ ID NO: 1 and SEQ ID: NO: 2 and complements thereof. The present invention also provides a soybean plant comprising a nucleic acid molecule selected from the group consisting of SEQ ID NO: 3 through SEQ ID NO: 10, fragments thereof, and complements of both. In one aspect, the soybean plant comprises 1 or 2 nucleic acid molecules selected from the group consisting of SEQ ID NO: 1 and SEQ ID NO: 2 and complements thereof. In another aspect, the soybean plant comprises 1 or 2 nucleic acid molecules selected from the group consisting of SEQ ID NO: 1 and SEQ ID NO: 2, fragments thereof, and complements of both. In a further aspect, the soybean plant comprises 1, 2, 3 or 4 nucleic acid molecules selected from the group consisting of SEQ ID NO: 3 through SEQ ID NO: 18 fragments thereof, and complements of both.

The present invention also provides a soybean plant comprising 1, or 2 SCN resistant loci where one or more alleles at one or more of their loci are selected from the group consisting of rhg1 and Rhg4. In one aspect, a soybean plant is provided comprising an rhg1. In another aspect, a soybean plant is provided comprising an Rhg4. In a further aspect, a soybean plant is provided comprising rhg1 and Rhg4. Such alleles may be homozygous or heterozygous.

The present invention also provides a soybean plant consisting of rhg1 and Rhg4 that exhibits nematode resistance and at least yield parity to susceptible varieties when cultivated under no, low, moderate or high nematode pressure.

Field populations of SCN are characterized as races or HG-types. A race designation reflects the ability of a particular field population to reproduce on a panel of a specified set of soybean germplasm, referred to as soybean host differentials. The test is conducted in monitored environments with controlled temperature and moisture conditions. After 30 days, the numbers of females on the roots of the indicator soybean lines are counted and compared to the number of females formed on a standard susceptible soybean line. The race test utilizes four indicator lines, Pickett, Peking, PI 88788 and PI 907663 and classifies SCN populations into 16 races (Riggs and Schmitt, J Nematol 20: 392-95, 1998). Peking is in the pedigree of Pickett and is the source of SCN resistance for Pickett. Therefore, Peking and Pickett often perform similarly in the race test. The HG (“HG” for Heterodera glycines) type test was developed to overcome the deficiencies associated with the race test, by eliminating the redundancy of Peking and Pickett and expanding the number soybean host differentials. The HG type test is performed similarly to the race test, but includes a broader panel of soybean host differentials. The HG-test utilized seven indicator lines: Peking (indicator line 1), PI 88788 (indicator line 2), PI 90763 (indicator line 3), PI 437654 (indicator line 4), PI 209332 (indicator line 5), PI 89772 (indicator line 6), and Cloud (indicator line 7) (Niblack et al. J. Nematol 34:279-88, 2002). The numbers of the HG type indicator soybean lines on which elevated SCN reproduction occurred are the numbers in the HG type designation. For example, an HG type 2.4 SCN population has elevated reproduction on the HG type indicator lines 2 and 4, P188788 and P1437654, respectively. Although the HG type test is the preferred method for SCN characterization for pathologists, breeders, and agronomists, SCN populations continue to be classified by both race and HG type classification.

Soybean lines were evaluated for SCN resistance in the greenhouse based on their response to a given SCN isolate. The SCN isolates were classified based on the race test or HG-type test. Both tests are performed similarly, but vary in the number of differentials. In the greenhouse bioassay, a soybean line replicated at least five times, is inoculated with nematode eggs and allowed to incubate for 28-35 days. At the end of this incubation period, cysts are extracted and counted under a microscope. The total number of cysts recovered from a soybean line is converted to a female index. The female index (%) is the number of cysts recovered from a given line, divided by the number of cysts recovered from the susceptible check. A line is declared resistant if the female index is less than 10% or susceptible if its female index is equal or greater than 10%. Thus, a given commercial variety is released as resistant or susceptible to any given SCN race or biotype based on the greenhouse assay only.

Field populations of SCN are diverse and heterogeneous. It is common to find many biotypes or races in a small patch in a field and their field distribution is highly heterogeneous. This is one of many difficulties involved in evaluating SCN disease reaction in the field. Field testing will aid in marker development (such as testing for yield drag), verification, and testing basic ecological hypotheses for furthering an understanding of the basic biological parameters influencing expression of resistance. Field testing has both advantages and disadvantages compared to greenhouse or growth chamber experiments. Field studies allow large plot sizes, seed increases, differing cultural practices, and natural interactions with other microorganisms and edaphic factors that will be common in the field. Field testing also requires an understanding that plant-parasitic nematodes occur in dynamic poly-specific communities that constantly respond to hosts, weather and climate, soil physical properties, other micro-fauna, and micro-flora. To date, there is no established methodology for SCN evaluation in the field.

In another aspect, the present invention provides a method for assaying soybean plants for yield in conjunction with nematode resistance, immunity, or susceptibility comprising: (a) determining the biotype of an nematode population (b) assaying density of nematode in field (c) cultivating field to maintain consistent nematode pressure, where the nematode pressure can be low (less than 500 eggs/100 cc soil) or high (greater than 500 eggs/100 cc soil) (d) cultivating soybean plants under both low and high nematode pressure and (d) evaluating the plants for nematode resistance and yield.

In another aspect, the present invention provides a method for breeding a soybean plant for yield and nematode susceptibility, partial resistance or resistance to nematodes comprising: (a) cultivating a soybean plant in a low infested and high infested nematode field nursery; (b) assessing the plant for susceptibility, partial resistance or resistance to nematodes; (d) assessing the plant for yield; and (d) selecting at least one soybean plant based on yield performance and nematode resistance.

Plants of the present invention can be a plant that is very resistant, resistant, substantially resistant, mid-resistant, comparatively resistant, partially resistant, mid-susceptible, or susceptible.

In a preferred aspect, the present invention provides a nematode resistant plant to be assayed for resistance or susceptibility to nematodes by any method to determine whether a plant is very resistant, resistant, substantially resistant, mid-resistant, comparatively resistant, partially resistant, mid-susceptible, or susceptible.

In yet another aspect, the invention provides a soybean plant that can show a comparative resistance compared to a non-resistant control soybean plant. In this aspect, a control soybean plant will preferably be genetically similar except for the nematode resistant allele or alleles derived from ‘Forrest’ in question. Such plants can be grown under similar conditions with equivalent or near equivalent exposure to the nematode. In this aspect, the resistant plant or plants has less than 25%, 15%, 10%, 5%, 2% or 1% of cysts compared to a non-resistant control soybean plant.

Rhg4 and rhg1 alleles of the present invention may be introduced into a SCN resistant line. An “elite line” is any line that has resulted from breeding and selection for superior agronomic performance.

Rhg4 and rhg1 alleles of the present invention may also be introduced into an elite soybean plant comprising one or more transgenes conferring herbicide tolerance, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistant, increased digestibility, industrial enzymes, pharmaceutical proteins, peptides and small molecules, improved processing traits, improved flavor, nitrogen fixation, hybrid seed production, reduced allergenicity, biopolymers, and biofuels among others. In one aspect, the herbicide tolerance is selected from the group consisting of glyphosate, dicamba, glufosinate, sulfonylurea, bromoxynil and norflurazon herbicides.

Rhg4 and rhg1 alleles can be introduced from any plant that contains that allele (donor) to any recipient soybean plant. In one aspect, the recipient soybean plant can contain additional SCN resistant loci. In another aspect, the recipient soybean plant can contain a transgene. In another aspect, while maintaining the introduced Rhg4 and rhg1 , the genetic contribution of the plant providing the Rhg4 and rhg1 can be reduced by back-crossing or other suitable approaches. In one aspect, the nuclear genetic material derived from the donor material in the soybean plant can be less than or about 50%, less than or about 25%, less than or about 13%, less than or about 5%, 3%, 2% or 1%, but that genetic material contains the Rhg4 and rhg1.

It is further understood that a soybean plant of the present invention may exhibit the characteristics of any relative maturity group. In an aspect, the maturity group is selected from the group consisting of MG 000, MG 00, MG 0, MG I, MG II, MG III, MG IV, MG V, MG VI, MG VII, MG VIII, MG IX and MG X.

An allele of a QTL can, of course, comprise multiple genes or other genetic factors even within a contiguous genomic region or linkage group, such as a haplotype. As used herein, an allele of a disease resistance locus can therefore encompass more than one gene or other genetic factor where each individual gene or genetic component is also capable of exhibiting allelic variation and where each gene or genetic factor is also capable of eliciting a phenotypic effect on the quantitative trait in question. In an aspect of the present invention the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present in the invention can denote a haplotype within a haplotype window wherein a phenotype can be disease resistance. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. As used herein, an allele is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus. Plants of the present invention may be homozygous or heterozygous at any particular rhg1 or Rhg4 for a particular polymorphic marker.

The present invention also provides for parts of the plants of the present invention. Plant parts, without limitation, include seed, endosperm, ovule and pollen. In a particularly preferred aspect of the present invention, the plant part is a seed.

The present invention also provides a container of seeds that exhibit SCN resistance and at least yield parity to commercial check varieties in which greater than 50%, 60%, 70%, 80%, 90%, 95%, or 99% of the seeds comprising rhg1 and Rhg4.

The container of seeds that exhibit SCN resistance and at least yield parity to commercial check varieties can contain any number, weight, or volume of seeds. For example, a container can contain at least, or greater than, about 10, 25, 50, 100, 200, 300, 400, 500, 600, 700, 80, 90, 1000, 1500, 2000, 2500, 3000, 3500, 4000 or more seeds. In another aspect, a container can contain about, or greater than about, 1 gram, 5 grams, 10 grams, 15 grams, 20 grams, 25 grams, 50 grams, 100 grams, 250 grams, 500 grams, or 1000 grams of seeds. Alternatively, the container can contain at least, or greater than, about 0 ounces, 1 ounce, 5 ounces, 10 ounces, 1 pound, 2 pounds, 3 pounds, 4 pounds, 5 pounds, 10 pounds, 15 pounds, 20 pounds, 25 pounds, or 50 pounds or more seeds.

Containers of seeds that exhibit SCN resistance and at least yield parity to commercial check varieties can be any container available in the art. For example, a container can be a box, a bag, a can, a packet, a pouch, a tape roll, a pail, or a tube.

In another aspect, the seeds contained in the containers of seeds that exhibit SCN resistance and at least yield parity to commercial check varieties can be treated or untreated seeds. In one aspect, the seeds can be treated to improve germination, for example, by priming the seeds, or by disinfection to protect against seed-born pathogens. In another aspect, seeds can be coated with any available coating to improve, for example, plantability, seed emergence, and protection against seed-born pathogens. Seed coating can be any form of seed coating including, but not limited to, pelleting, film coating, and encrustments.

Plants or parts thereof of the present invention may be grown in culture and regenerated. Methods for the regeneration of Glycine max plants from various tissue types and methods for the tissue culture of Glycine max are known in the art (See, for example, Widholm et al., In Vitro Selection and Culture-induced Variation in Soybean, In Soybean: Genetics, Molecular Biology and Biotechnology, Eds. Verma and Shoemaker, CAB International, Wallingford, Oxon, England (1996). Regeneration techniques for plants such as Glycine max can use as the starting material a variety of tissue or cell types. With Glycine max in particular, regeneration processes have been developed that begin with certain differentiated tissue types such as meristems, Cartha et al., Can. J. Bot. 59:1671-1679 (1981), hypocotyl sections, Cameya et al., Plant Science Letters 21: 289-294 (1981), and stem node segments, Saka et al, Plant Science Letters, 19: 193-201 (1980); Cheng et al., Plant Science Letters, 19: 91-99 (1980). Regeneration of whole sexually mature Glycine max plants from somatic embryos generated from explants of immature Glycine max embryos has been reported (Ranch et al., In Vitro Cellular & Developmental Biology 21: 653-658 (1985). Regeneration of mature Glycine max plants from tissue culture by organogenesis and embryogenesis has also been reported (Barwale et al., Planta 167: 473-481 (1986); Wright et al., Plant Cell Reports 5: 150-154 (1986).

The present invention also provides a SCN resistant soybean plant that exhibits at least similar yields to commercial check varieties selected for by screening for disease resistance or susceptibility in the soybean plant, the selection comprising introgressing genomic nucleic acids for the presence of a marker molecule that is genetically linked to an allele of a rhg1 and Rhg4 associated with disease resistance in the soybean plant.

Nucleic acid molecules or fragments thereof are capable of specifically hybridizing to other nucleic acid molecules under certain circumstances. As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, molecules are exhibit “complete complementarity” when every nucleotide of one of the molecules is complementary to a nucleotide of the other. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The nucleic-acid probes and primers of the present invention can hybridize under stringent conditions to a target DNA sequence. The term “stringent hybridization conditions” is defined as conditions under which a probe or primer hybridizes specifically with a target sequence(s) and not with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa 1984 Nucl. Acids Res. 12:203-213; and Wetmur et al. 1968 J. Mol. Biol. 31:349-370. Appropriate stringency conditions that promote DNA hybridization are, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

For example, hybridization using DNA or RNA probes or primers can be performed at 65° C. in 6×SSC, 0.5% SDS, 5×Denhardt's, 100 μg/mL nonspecific DNA (e.g., sonicated salmon sperm DNA) with washing at 0.5×SSC, 0.5% SDS at 65° C., for high stringency.

It is contemplated that lower stringency hybridization conditions such as lower hybridization and/or washing temperatures can be used to identify related sequences having a lower degree of sequence similarity if specificity of binding of the probe or primer to target sequence(s) is preserved. Accordingly, the nucleotide sequences of the present invention can be used for their ability to selectively form duplex molecules with complementary stretches of DNA, RNA, or cDNA fragments.

A fragment of a nucleic acid molecule can be any sized fragment and illustrative fragments include fragments of nucleic acid sequences set forth in SEQ ID NO: 1 through SEQ ID NO: 18 and complements thereof. In one aspect, a fragment can be between 15 and 25, 15 and 30, 15 and 40, 15 and 50, 15 and 100, 20 and 25, 20 and 30, 20 and 40, 20 and 50, 20 and 100, 25 and 30, 25 and 40, 25 and 50, 25 and 100, 30 and 40, 30 and 50, and 30 and 100. In another aspect, the fragment can be greater than 10 15, 20, 25, 30, 35, 40, 50, 100, or 250 nucleotides.

Additional genetic markers can be used to select plants with an allele of a QTL associated with SCN resistance of the present invention. Examples of public marker databases include, for example: Soybase, an Agricultural Research Service, United States Department of Agriculture.

Genetic markers of the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers, such as single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with alleles of a QTL of the present invention can be utilized.

In one embodiment, nucleic acid-based analyses for the presence or absence of the genetic polymorphism can be used for the selection of seeds in a breeding population. A wide variety of genetic markers for the analysis of genetic polymorphisms are available and known to those of skill in the art. The analysis may be used to select for genes, QTL, alleles, or genomic regions (haplotypes) that comprise or are linked to a genetic marker.

Herein, nucleic acid analysis methods are known in the art and include, but are not limited to, PCR-based detection methods (for example, TaqMan assays), microarray methods, and nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

For the purpose of QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes.

The genetic linkage of additional marker molecules can be established by a gene mapping model such as, without limitation, the flanking marker model reported by Lander et al. (Lander et al. 1989 Genetics, 121:185-199), and the interval mapping, based on maximum likelihood methods described therein, and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y.). Use of Qgene software is a particularly preferred approach.

A maximum likelihood estimate (MLE) for the presence of a marker is calculated, together with an MLE assuming no QTL effect, to avoid false positives. A logio of an odds ratio (LOD) is then calculated as: LOD=log₁₀ (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander et al. (1989), and further described by Arús and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak et al. 1995 Genetics, 139:1421-1428). Multiple regression methods or models can be also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen et al. (Jansen et al. 1994 Genetics, 136:1447-1455) and Zeng (Zeng 1994 Genetics 136:1457-1468). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp.195-204 (1994), thereby improving the precision and efficiency of QTL mapping (Zeng 1994). These models can be extended to multi-environment experiments to analyze genotype-environment interactions (Jansen et al. 1995 Theor. Appl. Genet. 91:33-3).

Selection of appropriate mapping populations is important to map construction. The choice of an appropriate mapping population depends on the type of marker systems employed (Tanksley et al., Molecular mapping in plant chromosomes. chromosome structure and function: Impact of new concepts J. P. Gustafson and R. Appels (eds.). Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

An F₂ population is the first generation of selfing. Usually a single F₁ plant is selfed to generate a population segregating for all the genes in Mendelian (1:2:1) fashion. Maximum genetic information is obtained from a completely classified F₂ population using a codominant marker system (Mather, Measurement of Linkage in Heredity: Methuen and Co., (1938)). In the case of dominant markers, progeny tests (e.g. F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g. disease resistance) or where trait expression is controlled by a QTL. Segregation data from progeny test populations (e.g. F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant markers evaluated in RIL populations provide more information per individual than either marker type in backcross populations (Reiter et al. 1992 Proc. Natl. Acad. Sci.(USA) 89:1477-1481). However, as the distance between markers becomes larger (i.e., loci become more independent), the information in RIL populations decreases dramatically.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts or mosaic of genomic regions from the donor parent. Backcross populations can be useful for mapping dominant markers if all loci in the recurrent parent are homozygous and the donor and recurrent parent have contrasting polymorphic marker alleles (Reiter et al. 1992). Information obtained from backcross populations, using either codominant or dominant markers, is less than that obtained from F₂ populations because one, rather than two, recombinant gametes are sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under introgression can be used as a mapping population. In mapping with NILs, only a portion of the polymorphic loci are expected to map to a selected region.

Bulk segregant analysis (BSA) is a method developed for the rapid identification of linkage between markers and traits of interest (Michelmore et al. 1991 Proc. Natl. Acad. Sci. (U.S.A.) 88:9828-9832). In BSA, two bulked DNA samples are drawn from a segregating population originating from a single cross. These bulks contain individuals that are identical for a particular trait (resistant or susceptible to particular disease) or genomic region but arbitrary at unlinked regions (i.e. heterozygous). Regions unlinked to the target region will not differ between the bulked samples of many individuals in BSA.

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pureline cultivar, etc). A cultivar is a race or variety of a plant species that has been created or selected intentionally and maintained through cultivation.

Selected, non-limiting approaches for breeding the plants of the present invention are set forth below. A breeding program can be enhanced using marker assisted selection (MAS) on the progeny of any cross. It is understood that nucleic acid markers of the present invention can be used in a MAS (breeding) program. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred aspect, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

Breeding lines can be tested and compared to appropriate standards in environments representative of the commercial target area(s) for two or more generations. The best lines are candidates for new commercial cultivars; those still deficient in traits may be used as parents to produce new populations for further selection.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Backcross breeding has been used to transfer genes for a simply inherited, highly heritable trait into a desirable homozygous cultivar or inbred line, which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have most attributes of the recurrent parent (e.g., cultivar) and, in addition, the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

Descriptions of other breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Manograph., 16:249, 1987; Fehr, “Principles of variety development,” Theory and Technique, (Vol. 1) and Crop Species Soybean (Vol. 2), Iowa State Univ., Macmillan Pub. Co., NY, 360-376, 1987).

An alternative to traditional QTL mapping involves achieving higher resolution by mapping haplotypes, versus individual markers (Fan et al. 2006 Genetics 172:663-686). This approach tracks blocks of DNA known as haplotypes, as defined by polymorphic markers, which are assumed to be identical by descent in the mapping population. This assumption results in a larger effective sample size, offering greater resolution of QTL. Methods for determining the statistical significance of a correlation between a phenotype and a genotype, in this case a haplotype, may be determined by any statistical test known in the art and with any accepted threshold of statistical significance being required. The application of particular methods and thresholds of significance are well with in the skill of the ordinary practitioner of the art.

It is further understood, that the present invention provides bacterial, viral, microbial, insect, mammalian and plant cells comprising the nucleic acid molecules of the present invention.

As used herein, a “nucleic acid molecule,” be it a naturally occurring molecule or otherwise may be “substantially purified”, if desired, referring to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

The agents of the present invention may be labeled with reagents that facilitate detection of the agent (e.g. fluorescent labels (Prober et al. 1987 Science 238:336-340; Albarella et al., European Patent 144914), chemical labels (Sheldon et al., U.S. Pat. No. 4,582,789; Albarella et al., U.S. Pat. No. 4,563,417), modified bases (Miyoshi et al., European Patent 119448).

Having now generally described the invention, the same will be more readily understood through reference to the following examples which are provided by way of illustration, and are not intended to be limiting of the present invention, unless specified.

### EXAMPLES

### Example 1

### Methods to Evaluate SCN Resistance and Yield

Soybean lines are evaluated for SCN resistance in the greenhouse based on their response to a given SCN isolate. SCN races are designated as Races 1 to 16 based on 4 differential lines Peking, Pickett, P188788 and PI 90763 with 3 being the most common race. Thus, a given commercial variety is released as resistant or susceptible to any given SCN race or biotype based on the greenhouse assay only. Field populations of SCN are diverse and heterogeneous. It is not uncommon to find many biotypes or races in a small patch in a field and their field distribution is highly heterogeneous. This is one of many difficulties involved in evaluating SCN disease in the field. To date, there is no established methodology for SCN evaluation in the field. A field screening assay was developed to evaluate SCN resistance in conjunction with yield.

Methods were developed to generate high and low SCN pressure environments and maintain consistent pressure through a season and from year-to-year. Locations were identified as suitable for the establishment of contrasting high and low SCN pressure field plots. These locations were identified based on the SCN disease pressures, history of soybean cropping, and soybean maturity zones. In each location, two plots were identified and were designated as high and low infested plots based on existing SCN disease pressures. SCN population densities were determined by extracting cysts from soil. SCN eggs were counted under the microscope. A high infested plot has moderate to high SCN infestation with greater than 500 eggs/100 cc of soil. A low infested plot has low SCN infestation with less than 500 eggs/100 cc of soil. The biotype of the SCN in the location was assayed with any standard test, such a race or Heterodera glycines (HG) type test. For example, the HG test measures the reproduction of the SCN population “HG type indicator” soybean lines with SCN resistance genes. The indicator lines are Peking (indicator line 1), PI 88788 (indicator line 2), PI 90763 (indicator line 3), PI 437654 (indicator line 4), PI 209332 (indicator line 5), PI 89772 (indicator line 6), and Cloud (indicator line 7). The numbers of the HG type indicator soybean lines on which elevated SCN reproduction occurred are the numbers in the HG type designation. For example, an HG type 2.4 SCN population has elevated reproduction on the HG type indicator lines 2 and 4, P188788 and P1437654, respectively. The test is conducted in monitored environments with controlled temperature and moisture conditions. After 30 days, the numbers of females on the roots of the 7 HG type indicator soybean lines are counted and compared to the number of females formed on a standard susceptible soybean line.

After SCN density and biotype was identified for a location, the plots were subdivided into four quadrants. Careful monitoring of each quadrant for every field in each site was performed annually to prevent the development of hot and cold spots.

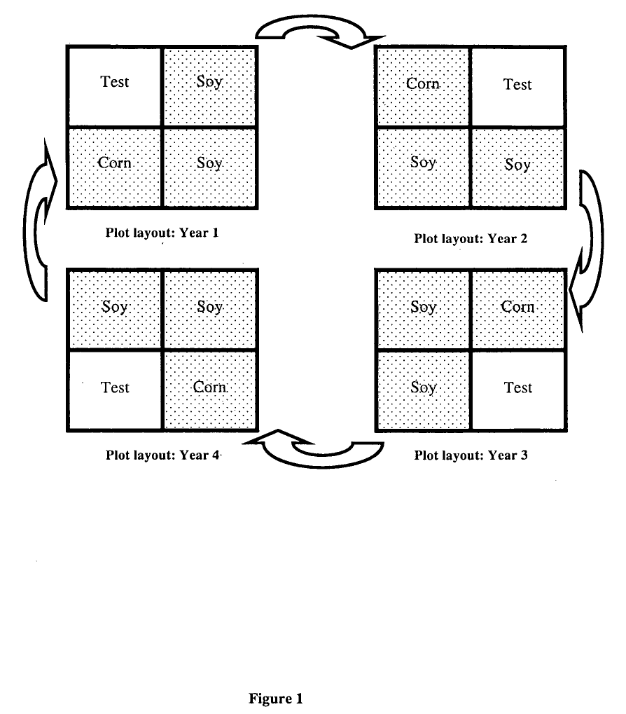

In the high infested field (FIG. 1), quadrant 1 was used as a test plot for the initial year and is planted to test entries. An herbicide and SCN-susceptible soybean was used as filler to plant rest of the plot that was not being used for testing. In this way, the SCN disease pressure was maintained. Quadrants 2 and 3 (maintenance plots) were planted with an herbicide and SCN-susceptible soybean, to maintain the high SCN disease pressure. A non-host crop like corn was planted to quadrant 4, to prevent SCN population from crashing which may occur with continuous planting to SCN-susceptible soybean. Quadrant 4 was the test plot the following season; it is important to maintain the SCN disease pressure and a crash or sudden decrease in SCN pressure due to continuous cropping with SCN-susceptible soybean. FIG. 1 shows the rotation of the quadrants.

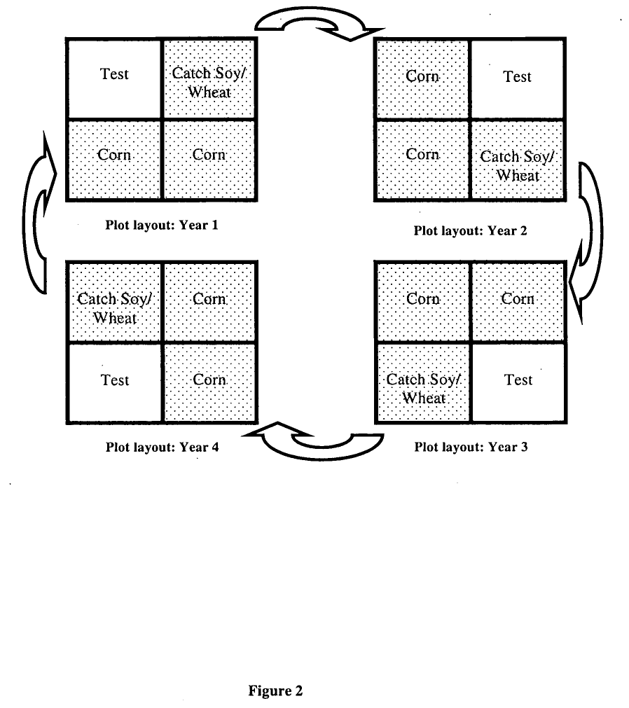

In the low infested field (FIG. 2), quadrant 1 was the test plot for the initial year. Quadrants 3-4 (maintenance plots) were devoted to a “sow and spray” method to reduce the SCN population in those plots. An herbicide- and SCN-susceptible variety was used as the catch crop and filler for these quadrants. The soy “catch crop” method promoted cyst hatching and the elimination of the nematodes just before they reach maturity to deplete the native SCN population in the soil. To clean up and maintain the low SCN level, a series of herbicide-susceptible and SCN-susceptible soybean “catch crop” was planted. The field was cultivated and planted with a high density of seed (herbicide and SCN susceptible variety). A spray application of herbicide was applied around 10 days after emergence (DAE), which is approximately at the V1-V2 stage. The soil was cultivated approximately 8 days after the spray application or when the plants were completely dead to avoid injury to the next cycle of planting of the susceptible soybean variety. Immediate planting after an herbicide application reduces the stand count due to root contact and translocation of herbicide. The cycle was repeated 3 to 4 times in the season to maximize SCN reduction. At the end of the growing season, the soil was cultivated and planted to another crop, such as oats or winter wheat, to overcome ‘fallow syndrome’. Fallow syndrome arises from the depletion of beneficial mycorrhizal fungi in the soil. The SCN pressure, either low or high, was consistent within the plots throughout the growing season and the subsequent spring (Tables 1-3).

TABLE 1
SCN egg densities throughout a growing season

| | |
|---|---|
| SCN | Eggs/100 cc soil |

| Infestation Type | Replication | Spring | Fall |
|---|---|---|---|
| High pressure | 1 | 2550.0 | 1800.0 |
| High pressure | 2 | 2325.0 | 1350.0 |
| High pressure | 3 | 1387.5 | 487.5 |
| High pressure | 4 | 537.5 | 512.5 |
| Low pressure | 1 | 300.0 | 237.5 |
| Low pressure | 2 | 312.5 | 375.0 |
| Low pressure | 3 | 433.3 | 450.0 |
| Low pressure | 4 | 962.5 | 225.0 |

TABLE 2
SCN egg densities throughout a growing season

| | |
|---|---|
| SCN | Eggs/100 cc soil |

| Infestation Type | Replication | Spring | Fall |
|---|---|---|---|
| High pressure | 1 | 2550.0 | 2700.0 |
| High pressure | 2 | 1775.0 | 2400.0 |
| High pressure | 3 | 2487.5 | 1900.0 |
| High pressure | 4 | 2337.5 | 2500.0 |
| Low pressure | 1 | 912.5 | 800.0 |
| Low pressure | 2 | 812.5 | 600.0 |
| Low pressure | 3 | 412.5 | 800.0 |
| Low pressure | 4 | 875.0 | 1000.0 |

TABLE 3
SCN egg densities throughout a growing season and subsequent Spring

| | | Eggs/100 cc soil | | |
|---|---|---|---|---|
| SCN | | Season 1 | | Season 2 |
| Infestation Type | Replication | Spring | Fall | Spring |
| High pressure | 1 | 925.0 | 500.0 | 1050.0 |
| High pressure | 2 | 1287.5 | 487.5 | 1900.0 |
| High pressure | 3 | 1466.667 | 512.5 | 3075.0 |
| High pressure | 4 | 766.6667 | 250.0 | 2500.0 |
| Low pressure | 1 | 212.5 | 187.5 | 200.0 |
| Low pressure | 2 | 212.5 | 100.0 | 175.0 |
| Low pressure | 3 | 137.5 | 237.5 | 200.0 |
| Low pressure | 4 | 475.0 | 150.0 | 375 |

### Example 2

### Assessing Yield Drag and Gains Utilizing High and Low Infested SCN Field Nurseries

With continued emphasis on developing and improving defensive traits for the commercial soybean seed program, there has been an increasing need to have field testing for evaluating plant responses to SCN and other nematodes. Field testing may aid in marker development, verification, and testing basic ecological hypotheses for furthering an understanding of the basic biological parameters influencing expression of resistance. Field studies allow large plot sizes, seed increases, differing cultural practices, and natural interactions with other microorganisms and edaphic factors that will be common in the field. Field testing also requires an understanding that plant-parasitic nematodes occur in dynamic poly-specific communities that constantly respond to hosts, weather and climate, soil physical properties, other micro-fauna, and micro-flora. Contrasting field nurseries with high and low SCN pressure (i.e., high and low infested fields) facilitated the identification of yield deficits and benefits within SCN resistant germplasm.

Three NIL populations were developed Accomac×MV0013, Accomac×MV0014 and Accomac×MV0024. Accomac is the SCN resistance source. Accomac has the resistance source ‘Forrest’ in its lineage. The segregation population was screened for the presence and absence of rhg1 derived from P188788, rhg1 derived from Forrest, and Rhg4 derived from Forrest. Haplotypes for rhg1 are described in Table 5. The SNP markers were developed by identifying polymorphisms within rhg1 . The progeny were separated into four classes using SNP markers based on the source of resistance. R8 has rhg1 derived P188788 and does not have Rhg4. R8RP has rhg1 derived P188788 and Rhg4 derived from Forrest. RP has rhg1 derived Forrest and does not have Rhg4. RPRP has rhg1 derived Forrest and Rhg4 derived from Forrest. Resistance to races 1, 3, 5, and 16 was assessed under a greenhouse assay (Table 4). Greenhouse assays were conducted to confirm the level of resistance for the genotype. The study evaluated resistance of the various gene combinations. RPRP had the broadest resistance and strongest resistance. RP, with rhg1 derive from Forrest alone, was susceptible to race 1 and 3, and moderately resistance to race 5.

TABLE 4
Resistance reaction of four classes (R8, R8RP, RP and
RPRP) of SCN resistant varieties to race 1, 3, 5, and 16

| | | Reaction* | | | |
|---|---|---|---|---|---|
| Gene Source | Class | Race 1 | Race 3 | Race 5 | Race 16 |
| rhg1(PI88788) | R8 | N/A | MR-R | N/A | MR-R |
| rhg1(PI88788) + | R8RP | S | MR-R | S | MR-R |
| Rhg4(Forrest-type) | | | | | |
| rhg1(Forrest-type) | RP | S | S | MS-MR | N/A |
| rhg1(Forrest-type) + | RPRP | R | R | MS-MR | N/A |
| Rhg4(Forrest-type) | | | | | |

*N/A = not applicable, MR = moderately resistant, MS = moderately susceptible, R = resistant, and S = susceptible

TABLE 5
Haplotypes for rhg1

| | SEQ ID NO | SEQ ID NO | SEQ ID NO |
|---|---|---|---|
| Marker Positions | 1 | 1 | 1 |

| | | | | | | |
|---|---|---|---|---|---|---|
| Haplotype | Source | Reaction* | 421 | 2561 | 3403 | Haplotype |

| | | | | | | |
|---|---|---|---|---|---|---|
| 1 | Peking | R | TT | GG | GG | TT/GG/GG |
| 2 | A3244 | S | AA | GG | GG | AA/GG/GG |
| 2 | Will | S | AA | GG | GG | AA/GG/GG |
| 3 | A2704 | S | AA | GG | CC | AA/GG/CC |
| 4 | Hutcheson | S | TT | AA | CC | TT/AA/CC |
| 4 | A1923 | S | TT | AA | CC | TT/AA/CC |
| 5 | Lee 74 | 5 | TT | GG | CC | TT/GG/CC |
| 5 | Essex | S | TT | GG | CC | TT/GG/CC |
| 5 | PI 88788 | R | TT | GG | CC | TT/GG/CC |

*R = resistant and S = susceptible

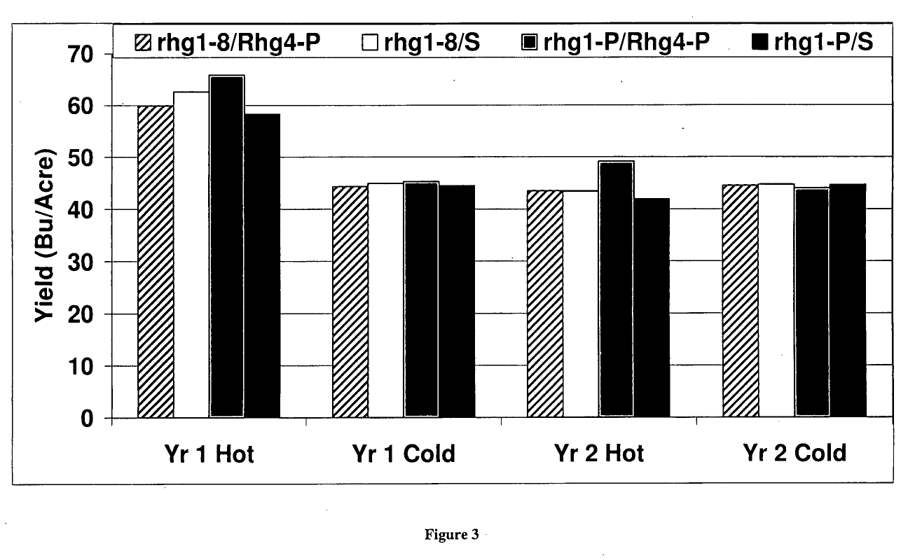

The strongest resistance to SCN was observed in plant with both rhg1 and Rhg4 derived from Peking. High and low infested field nurseries were used to evaluate yield impacts and SCN resistance. Plants with both rhg1 and Rhg4 from ‘Forrest’ derived from Peking had higher yield compared to plant lines with plant rhg1 derived from Peking alone or rhg1 derived from P188788 (FIG. 3). Commercially available SCN resistant varieties have higher yields compared to SCN susceptible varieties cultivated under high SCN pressure conditions, but have often lower yields compared to susceptible varieties cultivated under low SCN pressure conditions.

### Example 3

### Confirming Yield Parity and/or Gains with Forrest-Type SCN Resistance

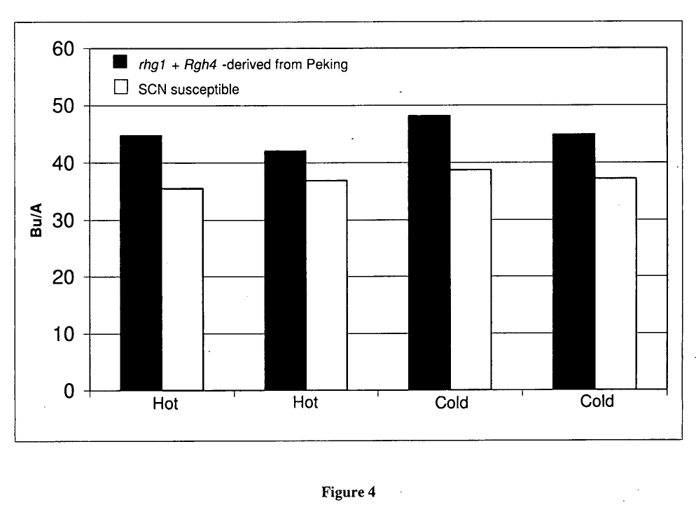

A population was developed by crossing MV0046 with MV0045. MV0045 was the source of resistance derived from ‘Forrest’. The progeny were genotyped for the rhg1 haplotypes and presence of Rhg4. The progeny were planted in high infestation and low infestation fields, evaluated for yield and SCN resistance. Progeny plants with rhg1 and Rhg4 from Forrest-type from Peking had higher yield than susceptible varieties cultivated under either high or low SCN pressure, suggesting soybeans with Forrest-type SCN resistance have a yield parity or gain compared to soybeans susceptible to SCN (Table 6; FIG. 4). Under low infestation conditions, soybeans with Forrest-type SCN resistance had 117% yield compared to susceptible soybeans. Under high infestation conditions, soybeans with Forrest-type SCN resistance had 114% yield compared to susceptible soybeans.

TABLE 6
SCN resistant soybeans with both Forrest-type rhg1 and Rhg4 have a
yield advantage compared with other SCN susceptible soybeans in both
low infestation and high infestation fields.

| | Field Treatment: | | | |
|---|---|---|---|---|
| | Low | High | | |
| | infestation | infestation | Resistance | Haplotype |
| Line | Yield (Bu/A) | Yield (Bu/A) | Class* | rhg1 |
| Progeny 1 | 45.94 | 45.09 | R | 1 |
| Progeny 2 | 46.34 | 39.25 | R | 1 |
| Progeny 3 | 48.57 | 45.92 | R | 1 |
| Progeny 4 | 49.16 | 45.14 | R | 1 |
| Progeny 5 | 47.07 | 50.70 | R | 1 |
| Progeny 6 | 51.05 | 49.03 | R | 1 |
| Progeny 7 | 49.08 | 41.33 | R | 1 |
| Progeny 8 | 48.90 | 46.23 | R | 1 |
| Progeny 9 | 48.80 | 41.36 | MR | 1 |
| Progeny 10 | 46.61 | 44.00 | R | 1 |
| Average for R* | 48.15 | 44.81 | R | |
| Progeny 11 | 36.56 | 33.33 | S | 4 |
| Progeny 12 | 37.57 | 34.92 | S | 4 |
| Progeny 13 | 37.52 | 33.58 | S | 4 |
| Progeny 14 | 38.10 | 37.04 | S | 4 |
| Progeny 15 | 36.76 | 40.60 | S | 4 |
| Progeny 16 | 42.54 | 37.12 | S | 4 |
| Progeny 17 | 36.81 | 30.99 | S | 4 |
| Progeny 18 | 42.10 | 32.68 | S | 4 |
| Progeny 19 | 40.66 | 39.80 | S | 4 |
| Average for S* | 38.74 | 35.56 | S | |
| Progeny 20 | 41.10 | 39.18 | MR | 1 |
| Progeny 21 | 42.08 | 44.97 | MR | 1 |
| Progeny 22 | 41.79 | 39.07 | MR | 1 |
| Progeny 21 | 41.04 | 41.05 | R | 1 |
| Progeny 23 | 45.35 | 43.48 | R | 1 |
| Progeny 24 | 41.13 | 37.09 | R | 1 |
| Progeny 22 | 50.66 | 39.94 | MR | 1 |
| Progeny 25 | 46.75 | 43.54 | R | 1 |
| Progeny 26 | 47.37 | 43.42 | R | 1 |
| Progeny 23 | 42.43 | 40.87 | R | 1 |
| Progeny 27 | 43.68 | 38.41 | R | 1 |
| Progeny 28 | 48.75 | 51.12 | R | 1 |
| Progeny 24 | 51.84 | 44.62 | MR | 1 |
| Average for R* | 44.92 | 42.06 | R | |
| Progeny 25 | 40.76 | 44.54 | S | 4 |
| Progeny 26 | 44.37 | 40.18 | S | 4 |
| Progeny 27 | 38.64 | 38.15 | S | 4 |
| Progeny 28 | 35.44 | 37.47 | S | 4 |
| Progeny 29 | 33.70 | 36.93 | S | 4 |
| Progeny 30 | 35.30 | 31.41 | S | 4 |
| Progeny 31 | 33.90 | 33.54 | S | 4 |
| Progeny 32 | 36.77 | 32.96 | S | 4 |
| Progeny 33 | 36.08 | 37.12 | S | 4 |
| Average for S* | 37.22 | 36.92 | S | |
| CV | 8.144 | 8.480 | | |
| LSD(.05) | 6.088 | 5.855 | | |
| F-Statistic | 8.420 | 8.874 | | |
| P-Value | 0.000 | 0.000 | | |
| Repeatability | 0.886 | 0.888 | | |
| Root MSE | 3.627 | 3.601 | | |

*MR = moderately resistant, R = resistant, S = susceptible

### Example 4

### Utilization of Molecular Markers Associated with Nematode Resistance and Yield to Facilitate Introgression of a Trait

If a variety possesses a desirable trait, such as nematode resistance and yield, it may readily be transferred to other varieties by crossing. Molecular markers associated with nematode resistance and at least yield parity to susceptible plants irrespective of nematode infestation levels, allows breeders to cross with parents with agronomically elite phenotypes, select seed of the cross based on the presence of the trait, and subsequently select for agronomically elite phenotype. It is within the scope of this invention to utilize the methods and compositions for preferred trait integration of nematode resistance and yield irrespective of nematode infestation level. It is contemplated by the inventors that the present invention will be useful for developing commercial varieties with nematode resistance and high yield irrespective of nematode infestation level.

Having illustrated and described the principles of the present invention, it should be apparent to persons skilled in the art that the invention can be modified in arrangement and detail without departing from such principles. We claim all modifications that are within the spirit, scope and concept of the appended claims.

What is claimed is:

1. A method of soybean breeding comprising the steps of:
a. crossing a first soybean having Forrest-type SCN resistance with a second soybean to create a segregating population; and
b. selecting a progeny plant comprising Forrest-type SCN resistance alleles of rhg1 and Rhg4, wherein said selected progeny plant is capable of SCN resistance under field conditions while having a grain yield at least equal to that of a SCN susceptible progeny plant irrespective of growing field SCN infestation levels.

2. The method of claim 1, wherein the selected progeny plant is capable of producing a grain yield at least about 5% higher than that of the SCN susceptible progeny plant irrespective of growing field nematode infestation levels.

3. The method of claim 1, wherein said first soybean having Forrest-type SCN resistance is a plant derived from the soybean variety Accomac or MV0045.

4. The method of claim 1, wherein said Forrest-type SCN resistance alleles of rhg1 and Rhg4 are detectable by interrogating the genomic DNA of the progeny plants for the presence of polymorphisms in the sequences of SEQ ID NOs: 1 and 2.

5. The method of claim 1, wherein said selected progeny plant comprises haplotype 1 for SEQ ID NO: 1 at the rhg1 locus.

6. A soybean plant comprising introgressed Forrest-type SCN resistance alleles of rhg1 and Rhg4, wherein said soybean plant is capable of SCN resistance under field conditions while maintaining at least yield parity to a commercial check variety irrespective of growing field nematode infestation levels.

7. The soybean plant of claim 6, wherein said soybean plant comprises haplotype 1 for SEQ ID NO: 1 at the rhg1 locus.

8. The soybean plant of claim 6, wherein said soybean plant is capable of producing a grain yield at least about 5% higher than that of a commercial check variety under non-, low-, moderate-, or high-infestation levels.

9. The soybean plant of claim 6, wherein said commercial check variety is the soybean variety AG2703 or DKB23-51.

10. The soybean plant of claim 6, wherein said soybean plant further comprises a transgenic trait.

11. The soybean plant according to claim 10, wherein the transgenic trait confers to the soybean plant a preferred property selected from the group consisting of herbicide tolerance, increased yield, insect control, fungal disease resistance, virus resistance, nematode resistance, bacterial disease resistance, mycoplasma disease resistance, altered fatty acid composition, altered oil production, altered amino acid composition, altered protein production, increased protein production, altered carbohydrate production, germination and seedling growth control, enhanced animal and human nutrition, low raffinose, drought and/or environmental stress tolerance, altered morphological characteristics, increased digestibility, industrial enzymes, pharmaceutical proteins, peptides and small molecules, improved processing traits, improved flavor, nitrogen fixation, hybrid seed production, reduced allergenicity, biopolymers, biofuels, and any combination of these.

12. A method of selecting high yielding soybean plants comprising:
a. providing a population of soybean plants:
b. exposing said population of soybean plants to moderate to high levels of SCN infestation; and
c. selecting a SCN resistant plant comprising haplotype 1 for SEQ ID NO: 1 at the rhg1 locus, wherein progeny of said selected SCN resistant plant is capable of producing a grain yield at least that of a commercial check variety irrespective of growing field nematode infestation levels.

13. The method of claim 12, wherein the progeny of said selected SCN resistant plant is capable of producing a grain yield at least 5% higher than that of a commercial check variety irrespective of growing field nematode infestation levels.

14. The method of claim 12, wherein said commercial check variety is the soybean variety AG2703 or DKB23-51.

15. A method for assessing nematode resistant and susceptible plant cultivar yield response comprising the steps of:
a. establishing at least two field nurseries with variable pressures of nematode infestation;
b. maintaining in each nursery a relatively consistent infestation pressure through a growing season and from season-to-season;
c. planting a test entry cultivar in said at least two field nurseries; and
d. measuring yield performance of said test entry from said at least two field nurseries.

16. The method of claim 15, wherein at least one test entry is selected for its yield performance under different nematode infestation pressures.

17. The method of claim 15, wherein at least one test entry is selected for its yield performance under different nematode infestation pressures.

18. The method of claim 15, wherein step b) is accomplished by cultivating and eliminating nematode susceptible plants cultivated proximal to a test plot.

19. The method of claim 16, wherein nematode susceptible plants are planted, cultivated, and eliminated at least three times during a growing season.

20. The method of claim 15, wherein the nematode infestation pressure in at least one field nursery is maintained by cultivating nematode susceptible plants and non-nematode host plants proximal to a test plot.

21. The method of claim 15, wherein the nematode is one selected from the group consisting of Heterodera sp. such as soybean cyst nematode (Heterodera glycines), Belonolaimus sp. such as sting nematode (Belonolaimus longicaudatus), Rotylenchulus sp. such as reniform nematode (Rotylenchulus reniformis), Meloidogyne sp. such as southern root-knot nematode (Meloidogyne incognita), peanut root-knot nematode (Meloidogyne arenaria), and the Javanese root-knot nematode (Meloidogyne javanica).

22. A method of promoting a soybean variety comprising providing information that said soybean variety is capable of nematode resistance and high yield.

23. The method of claim 22, wherein said information further comprises a high soybean yield irrespective of nematode infestation pressure.

24. The method of claim 22, wherein said information further comprises the origin of nematode resistance in said soybean variety, wherein said origin of nematode resistance is “Forrest”, “Peking”, or “Accomac”.

25. The method of claim 22, wherein the information is disseminated by an oral or visual medium selected from the group consisting of television, film, video, radio, extension presentations, oral presentations, print, newspapers, magazines, technical bulletins, extension bulletins, packaging, seed bags, bag tags, brochures, photography, electronic, internet, blogs, and e-mail.
